(19) United States
(12) Patent Application Publication
(10) Pub. No.: US 2022/0125339 A1
(43) Pub. Date: Apr. 28, 2022

(54) DEVICE AND SYSTEM FOR MEASURING RANGE OF MOTION OF ANKLE, AND METHODS FOR USING THE SAME

(71) Applicant: Mohsen ABASSI, Doha (QA)

(72) Inventor: Mohsen ABASSI, Doha (QA)

(21) Appl. No.: 17/509,839

(22) Filed: Oct. 25, 2021

Related U.S. Application Data
(60) Provisional application No. 63/104,810, filed on Oct. 23, 2020.

(51) Int. Cl. A61B 5/11; A61B 5/00

(52) U.S. Cl. CPC A61B 5/1121; A61B 2562/0247; A61B 5/4595; A61B 5/1126

(57) ABSTRACT

The device for measuring range of motion of an ankle provides for angular measurement of the range of motion of a patient's ankle, and includes a foot rest having a base, a pair of sidewalls and an inclined upper surface. A foot retainer releasably receives the patient's foot. A mounting plate is slidably mounted to one of the pair of sidewalls. The lower end of an elongated rod is rotatably mounted to the mounting plate. An inclinometer is secured to the elongated rod. A retaining bar is secured to the elongated rod, adjacent its upper end. The retaining bar is adapted for positioning adjacent a calf muscle of the patient when the patient's foot is received in the foot retainer.

### CROSS-REFERENCE TO RELATED APPLICATIONS

The present application claims priority to and the benefit of U.S. Provisional Application No. 63/104,810, filed on Oct. 23, 2020, the entirety of which is herein incorporated by reference.

### FIELD

The devices and systems for measuring range of motion of an ankle, and methods for using the same are described herein. In some embodiments, the devices for measuring range of motion of an ankle described herein are for both side-to-side and vertical foot movements.

### BACKGROUND

The global musculoskeletal diseases treatment market has been increasing consistently, due to the rising prevalence of musculoskeletal disorders, which are mainly associated with sports, occupational injuries, pathologic conditions, and age-related dysfunction. Examples of the most common musculoskeletal diseases include back pain, rheumatoid arthritis, osteoarthritis, osteoporosis, septic arthritis, gout, myasthenia gravis, systemic lupus erythematous (SLE), and other related pathologies. Musculoskeletal system is a diagnosis and management of all acute and chronic conditions affecting the musculoskeletal system, i.e., muscles, bones, tendons, joints and ligaments and treatment of non-surgical lesions of the musculoskeletal system.

Evaluation of range of motion (ROM) is integral to the assessment of the musculoskeletal system. The ankle joint is a common site of musculoskeletal pathology. Measurement of its functional range of motion is a primary indicator for rehabilitation outcomes in therapy settings. Clinical and epidemiological studies have identified limitation in ankle dorsiflexion and increased ankle stiffness as key contributors to the evolution of foot and ankle pathology. Accurate measurement of relatively small changes in ankle ROM is crucial to precisely identify impairments. Yet, conventional methods for quantifying ankle ROM and stiffness lack accurate and robust measurement techniques, thus making it difficult to assess any functional consequences of relatively small changes in ankle ROM.

Furthermore, beyond the practical difficulties of applying current techniques in a clinic, failure to stabilize the foot and the localized application of force may not adequately replicate loading of the foot during functional tasks.

Computerized approaches have been proposed to isolate ankle joint motion and measure the applied external torque, thus enabling measurement of both ankle ROM and ankle stiffness (e.g., changes in ankle dorsiflexion angle as a function of applied external torque). However, the increased accuracy of these more sophisticated measurement approaches comes at high financial cost. These systems also tend to be difficult to transport and complicated to use, raising questions as to their clinical usefulness and cost effectiveness. Most importantly, a few of these ROM devices have demonstrated the less-than-optimal reliability and validity of ankle ROM measurements.

An ankle includes three joints, namely the ankle joint proper or talocrural joint, the subtalar joint, and the inferior tibiofibular joint. The full ROM of a person's foot about the ankle is defined by the range of motion of each of these three joints, particularly side-to-side foot motion (i.e., eversion and inversion) and vertical foot motion (i.e., dorsiflexion and plantar flexion). In order to diagnose angle injury, most measurements of ankle range of motion are only taken in an up and down direction (i.e., vertical angular movement). Only measuring dorsiflexion and plantar flexion is equivalent to leaving at least half of potentially valuable data unmeasured. Thus, there remains a need for devices for measuring range of motion of an ankle solving the aforementioned problems.

### SUMMARY

Devices for measuring range of motion of an ankle described herein provide for angular measurement of the range of motion of a patient's ankle. In some embodiments, devices for measuring range of motion of an ankle comprise a foot rest having a base, a pair of sidewalls and an inclined upper surface. In other embodiments, devices of the present disclosure comprise a foot retainer which can be secured to the inclined upper surface of the foot rest for releasably receiving the patient's foot. The foot retainer can be rotatably secured to the inclined upper surface. In other embodiments, devices of the present disclosure comprise a mounting plate which can be slidably mounted on one of the pair of sidewalls, such that the mounting plate can be selectively slidable along a direction parallel to the inclined upper surface for proper positioning with respect to the patient's foot and ankle. The mounting plate can be slidably mounted to the foot rest in any suitable manner, such as, for example, but not limited to, by a pair of brackets secured to the foot rest for slidably receiving the mounting plate.

In some embodiments, devices for measuring range of motion of an ankle comprise an elongated rod, having opposed upper and lower ends. The lower end can be rotatably mounted onto the mounting plate. Further, an inclinometer, such as a digital inclinometer, angle sensor or the like, can be secured to the elongated rod for measuring an angular displacement of the elongated rod with respect to the inclined upper surface of the foot rest. A measuring member can be further fixedly secured to the mounting plate. The measuring member has indicia formed thereon indicating an angular displacement of the elongated rod, such that the elongated rod and the measuring member form a conventional goniometer. In some embodiments, the inclinometer can perform the measurement of the patient's range of motion, and the goniometer can be used for calibrating the inclinometer.

Embodiments further comprise a retaining bar secured to the elongated rod, adjacent the upper end thereof, with the retaining bar extending substantially orthogonal to the elongated rod. The retaining bar can be adapted for positioning adjacent a leg of the patient when the patient's foot is received in the foot retainer. The retaining bar can be slidably mounted on the elongated rod for selectively adjusting a height of the retaining bar with respect to the foot rest, allowing the device for measuring range of motion of an ankle to be used by patients having varying heights and body types.

These and other features of the present disclosure will become readily apparent upon further review of the following specification and drawings.

### BRIEF DESCRIPTION OF THE DRAWINGS

The following figures are included to illustrate certain aspects of the present disclosure and should not be viewed as exclusive embodiments. The subject matter disclosed is capable of considerable modifications, alterations, combinations, and equivalents in form and function, as will occur to one having ordinary skill in the art and having the benefit of this disclosure.

FIG. 1 is a perspective view of a device for measuring range of motion of an ankle described herein.

FIG. 2 is a perspective view of the device for measuring range of motion of an ankle in a first configuration.

FIG. 3 is a perspective view of the device for measuring range of motion of an ankle in an alternative configuration.

FIG. 4 illustrates portability and packaging concepts.

FIG. 5 illustrates sensors to measure the strength.

### DETAILED DESCRIPTION

The present disclosure relates to devices and systems for measuring range of motion of an ankle, and methods for using the same. In some embodiments, the devices for measuring range of motion of an ankle described herein are for both side-to-side (i.e., eversion and inversion) and vertical foot movements (i.e., dorsiflexion and plantar flexion).

As discussed above, there is growing demand for an ankle ROM measurement system with higher measurement accuracy than the conventional methods. The ankle comprises three joints, namely the ankle joint proper or talocrural joint, the subtalar joint, and the inferior tibiofibular joint. The full ROM of a person's foot about the ankle is defined by the range of motion of each of these three joints, particularly side-to-side foot motion (i.e., eversion and inversion) and vertical foot motion (i.e., dorsiflexion and plantar flexion). In order to diagnose angle injury, most measurements of ankle range of motion are only taken in an up and down direction (i.e., vertical angular movement). Only measuring vertical angular movement is equivalent to leaving at least half of potentially valuable data unmeasured. Thus, a device for measuring range of motion of an ankle solving the aforementioned problems is desired.

The present disclosure provides devices, systems and methods for measuring range of motion of an ankle with accurate measurement of the ankle joint in all cardinal planes (e.g., angular measurement of the range of motion of a patient's ankle), while simultaneously allowing for adjustment of the axis of rotation by variable placement of the center of rotation. Devices of the present disclosure enable controlled measurement of said ranges of motion while the patient's ankle is fully weight bearing (or simulative of full weight bearing position). Hence, the present disclosure provides an accurate assessment of any alteration in ankle flexibility in a cost-effective manner.

Furthermore, the present disclosure relates to articulating joints and a suspension system that allows for improved portability of the device for practical packaging, transfer, and carrying of the device.

All numerical values within the detailed description and the claims herein are modified by “about” or “approximately” with respect to the indicated value, and take into account experimental error and variations that would be expected by a person having ordinary skill in the art.

The term “and/or” as used in a phrase such as “A and/or B” herein is intended to include “A and B,” “A or B,” “A,” and “B.”

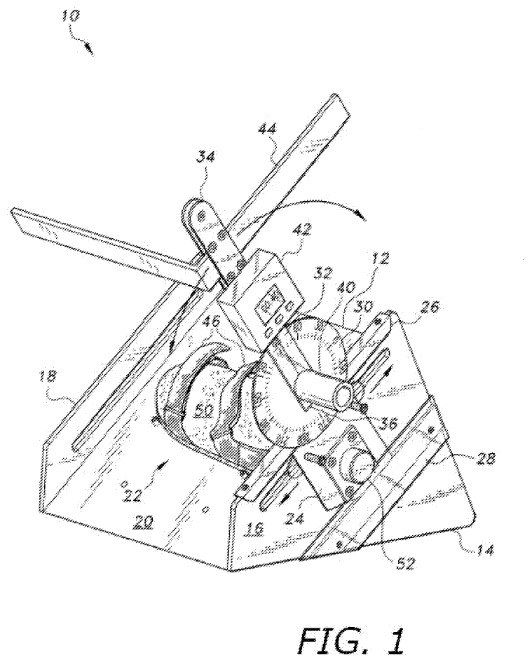

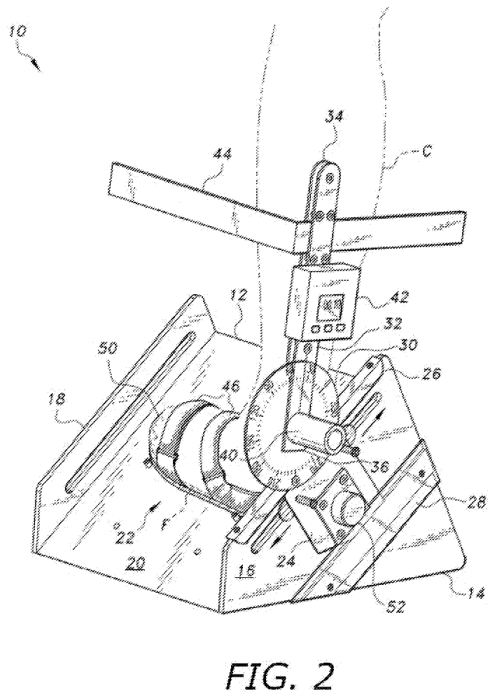

The device for measuring range of motion of an ankle 10 provides for angular measurement of the range of motion of a patient's ankle. As shown in FIGS. 1 and 2, the device for measuring range of motion of an ankle 10 comprises foot rest 12 having base 14, at least one sidewall, such as pair of sidewalls 16 and 18, and an inclined upper surface 20. Inclined upper surface 20 of foot rest 12 is configured to allow the patient to position his or her foot (F) at a horizontal angle, allowing for measurement of side-to-side motion and vertical range of motion The device for measuring range of motion of an ankle 10 can be configured to measure of a full functional range of motion of the ankle, including, but is not limited to, contributions from both the subtalar joint, and the talocrural joint. It should be understood that the overall contouring and relative dimensions of foot rest 12 are shown for exemplary purposes only. Inclined upper surface 20 is inclined at about 40° with respect to base 14, which is adapted for sitting on a support surface, such as a horizontal floor surface. In some embodiments, inclined upper surface 20 is inclined at about 10° or more, such as about 15° or more, such as about 20° or more, such as 25° or more, such as about 30° or more, such as about 35° or more, such as about 40° or more, such as about 45° or more. In other embodiments, inclined upper surface 20 is inclined at about 60° or less, such as about 55° or less, such as about 50° or less, such as about 45° or less, such as about 40° or less, such as about 35° or less, such as about 30° or less, such as about 25° or less.

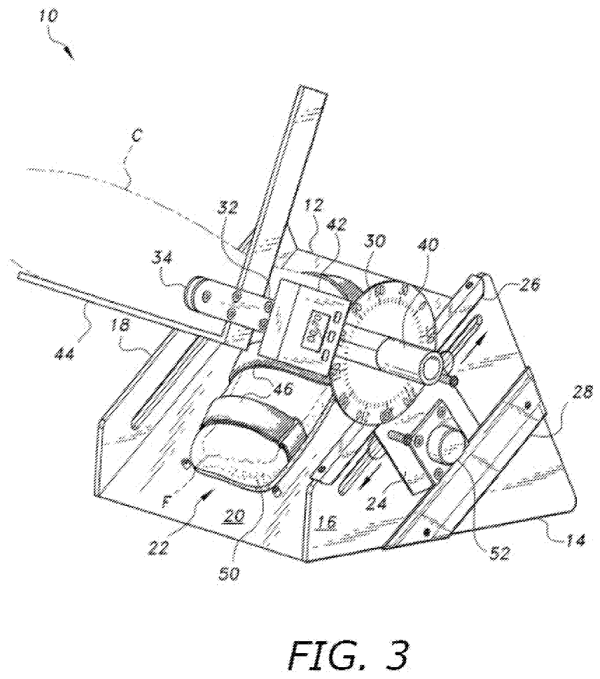

Foot retainer 22 is secured to inclined upper surface 20 of foot rest 12 for releasably receiving the patient's foot (F). Foot retainer 22 is rotatably secured to inclined upper surface 20 to allow foot rest 12 to be rotated 360°. In some embodiments, the foot retainer can be detachably secured to the foot rest to permit repositioning of the foot retainer in this manner. The foot retainer can be positioned for measuring a side or horizontal bending movement of the ankle joint, as illustrated in FIG. 2 or for measuring a vertical bending movement of the ankle joint, as illustrated in FIG. 3. Foot retainer 22 comprises pad 50 or a contoured mount for comfortably supporting the sole of the patient's foot. At least one strap 46 can be provided for releasably securing the patient's foot during measurement. It should be understood that any suitable type of pad or retainer can be used in the construction of foot retainer 22, and that straps 46 are shown for exemplary purposes only, and foot (F) can be secured to foot retainer 22 by any suitable releasable fixture or fastener.

Mounting plate 24 is slidably mounted onto one of the pair of sidewalls. In the examples of FIGS. 1-3, mounting plate 24 is shown slidably mounted to sidewall 16, although it should be understood that mounting plate 24 could be slidably mounted to sidewall 18 with the device for measuring range of motion of an ankle 10 operating in a substantially identical manner. As shown, mounting plate 24 is selectively slidable along a direction parallel to inclined upper surface 20 for proper positioning with respect to the patient's foot and ankle. Mounting plate 24 can be selectively held in a desired position by tightening knob 52, or through the usage of any suitable type of engaging member, locking device, frictional fit or the like. It should be understood that mounting plate 24 can be slidably mounted to foot rest 12 in any suitable manner, such as, for example, by a pair of brackets 26, 28 secured to foot rest 12 for slidably receiving mounting plate 24.

In some embodiments, the devices further comprise an elongated rod 32, having opposed upper and lower ends, 34 and 36 comprises lower end 36 being rotatably mounted to mounting plate 24, using knob 40. Inclinometer 42, such as a digital inclinometer, angle sensor or the like, is secured to elongated rod 32 for measuring an angular displacement of elongated rod 32 with respect to inclined upper surface 20 of foot rest 12. Measuring member 30 can be further secured to mounting plate 24. Measuring member 30 has indicia formed thereon indicating an angular displacement of elongated rod 32, such that the elongated rod 32 and the measuring member 30 form a device that measures an angle or permits the rotation of an object to a definite position (e.g., goniometer). Measuring range can be from 0° to 360°, such as 0° to 340°, such as 0° to 320°, such as 0° to 300°, such as 0° to 280°, such as 0° to 260°, such as 0° to 240°, such as 0° to 220°, such as 0° to 200°, such as 0° to 180°, such as 0° to 160°, such as 0° to 140°, such as 0° to 120°, such as 0° to 100°. It should be understood that the inclinometer can perform the measurement of the patient's range of motion, and the goniometer can be used for calibrating inclinometer 42.

Retaining bar 44 is secured to elongated rod 32, adjacent upper end 34 thereof, with retaining bar 44 extending substantially orthogonal to elongated rod 32. It should be understood that the substantially L-shaped retaining bar 44 is shown for exemplary purposes only. The retaining bar can be configured into any suitable shape. Retaining bar 44 is adapted for positioning adjacent or proximate a calf muscle (C) of the patient when the patient's foot is received in the foot retainer 22 (about 20 cm below the tuberosity of the tibia in an average adult). Retaining bar 44 is slidably mounted on elongated rod 32 for selectively adjusting a height of retaining bar 44 with respect to foot rest 12, allowing the device for measuring range of motion of an ankle 10 to be used by patients having varying heights and body types.

In some embodiments, a method of using the devices described herein, the patient places his or her foot on foot retainer 22 and lowers his or her lower leg as much as possible; i.e., the patient stretches in the direction of motion being measured until he or she reaches the maximum tolerable angle. Retaining bar 44 is then lowered to the level of the leg while the patient's foot is held in foot retainer 22. The inclinometer 42 measures the degree to which the patient can bend his or her ankle, as indicated by the level of the lowered leg. For plantar flexion movement, the foot is positioned on foot retainer 22 such that the toes point toward the base of foot rest 12, as shown in FIG. 3. Once in this position, the patient stretches forward to measure plantar flexion movement. To measure dorsiflexion movement, foot retainer 22 is turned 180° from the position shown in FIG. 3 and the same foot is positioned heel first onto foot retainer 22, i.e. with the patient facing away from foot rest 12, such that the toes point toward the top edge of foot rest 12. Once in this position, the patient stretches forward to measure dorsiflexion movement. For measuring side-to-side motion, the foot is positioned across foot rest 12, as shown in FIG. 2, and the patient stretches to one side, away from foot rest 12. To measure the range of motion for the opposite side of the same ankle, the patient can rotate the foot retainer 180° and reposition his or her foot accordingly.

It should be understood that devices 10 described herein can be used in combination with additional measurement devices or aids for precision measurement. For example, one or more foot plate sensors can be positioned under the foot of the patient to compensate for any unwanted motion of the patient, such as elevation of part of the foot during extreme bending of the ankle. As another example, an additional laser pointer or the like can be added, fixed to elongated rod 32, to identify the internal axis of the measured ankle that joins two malleoli with respect to one another. Such a laser pointer could be received within the housing of joint, for example.

The device for measuring range of motion of an ankle 10 allows for accurate measurement of the ankle joint in all cardinal planes, while simultaneously allowing for adjustment of the axis of rotation by variable placement of the center of rotation. Device 10 is configured for controlled measurement of these ranges of motion while the patient's ankle is fully weight bearing (or simulative of full weight bearing position). This provides for an accurate assessment of any alteration in ankle flexibility. As described above, device 10 is configured to enable measurement of the ankle in a functional manner, enabling it to move along three-dimensional x, y, and z axes, e.g., in a forward direction and opposing side directions.

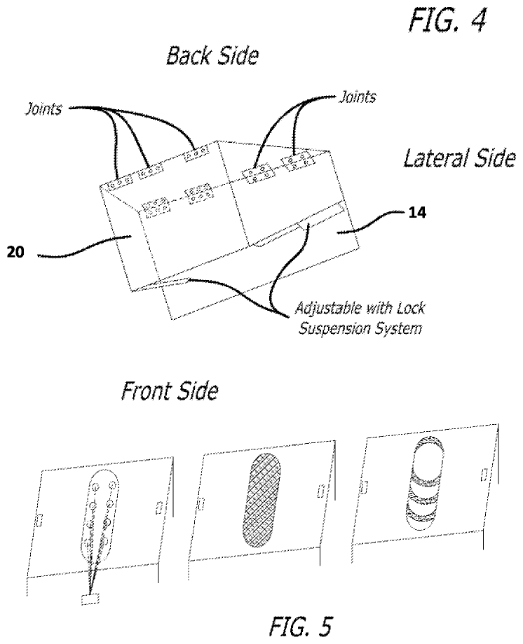

The present disclosure relates to articulating joints and a suspension system that allows for improved portability of the device for practical packaging, transfer, and carrying of the device. FIG. 4 illustrates at least one embodiment of the design, wherein inclined upper surface 20 is attached to the rear surface through a first set of articulating joints, allowing the surfaces to pivot about the joint toward and away from each other. The rear surface attaches to base 14 through a second set of articulating joints, again allowing the surfaces to pivot about the joint toward and away from each other. Inclined upper surface 20 attaches to base 14 through a suspension system affixed to the base. During use, the user positions his foot on the upper surface and applies pressure to his foot to lower inclined upper surface 20 toward the base through the suspension system. Once the user reaches the maximum angle of the motion to be measured, the suspension system locks in place and the measurement is taken.

In some embodiments, devices of the present disclosure comprise sensors on foot retainer 22 to measure ankle muscle strength. Foot retainer 22 includes a foot plate with a plurality of straps 46 for securing the patient's foot. As shown in FIG. 5 (left), a first set of sensors can be positioned along a foot area of foot rest 12. A cover material can be secured over the sensors to secure the sensors in place and allow for the transmission of pressure, as shown in FIG. 5 (middle). Referring to FIG. 5 (right), a second set of sensors is positioned transversally and inferiorly along the internal surface of the straps. In some embodiments, sensors can be small, thin pressure sensors. Output reading components from the first and second sets of sensors are received by an acquisition data system. For example, the output components from the first and second sets of sensors can communicate with a processor of a patient device, such as a mobile phone, tablet, computer or the like, or with a processor of a clinician device, such as a mobile phone, tablet, computer or the like.

In some embodiments, methods of using devices are described herein. In other embodiments, methods for measuring range of motion of an ankle comprise: placing a foot of a subject on a surface of a foot rest, wherein the foot rest comprises a base, at least one sidewall, and an inclined upper surface, and wherein a foot retainer is secured to the inclined upper surface of the foot rest for releasably receiving the foot of the subject; measuring a side or horizontal bending movement of the ankle joint; measuring a vertical bending movement of the ankle joint; and obtaining information representative of one or more data sets from an acquisition data system during one or more physical actions performed by the subject.

One or more illustrative embodiments are presented herein. Not all features of a physical implementation are described or shown in this application for the sake of clarity. It is understood that in the development of a physical embodiment of the present disclosure, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for one of ordinary skill in the art and having benefit of this disclosure.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to one having ordinary skill in the art and having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present disclosure. The embodiments illustratively disclosed herein suitably may be practiced in the absence of any element that is not specifically disclosed herein and/or any optional element disclosed herein.

It is to be understood that the present disclosure is not limited to the embodiments described above, but encompasses any and all embodiments within the scope of the following claims.

What is claimed is:

1. A device for measuring range of motion of an ankle, the device comprising:
a foot rest comprising an inclined upper surface;
a base;
at least one sidewall,
a foot retainer configured to secure a foot of a subject to the inclined upper surface of the foot rest for releasably receiving the foot of the subject;
a mounting plate, wherein the mounting plate is slidably mounted to the at least one sidewall;
an inclinometer, wherein the inclinometer is secured to an elongated rod;
a retaining bar, wherein the retaining bar is secured to the elongated rod adjacent its upper end, wherein the retaining bar is configured for positioning adjacent a calf muscle of the patient when the patient's foot is received in the foot retainer;
at least one strap disposed on the surface of the foot rest;
a first set of one or more sensors disposed on the surface of the foot rest;
a second set of one or more sensors disposed transversally and inferiorly along an internal surface of the at least one strap;
a first output component of the first set of the one or more sensors;
a second output of the second set of the one or more sensors; and
an elongated rod having opposed upper end and lower end, wherein the lower end is rotatably mounted to the mounting plate.

2. The device of claim 1, further comprising a measuring member, wherein the elongated rod and the measuring member form a goniometer.

3. The device of claim 1, further comprising:
a first set of one or more articulating joints configured to connect the inclined upper surface to the rear surface of the device, wherein the first set of one or more articulating joints allows the inclined upper and the rear surface to pivot toward and away from each other.

4. The device of claim 1, further comprising:
a second set of one or more articulating joints configured to connect the rear surface of the device to the base, wherein the second set of one or more articulating joint allows the rear surface and the base to pivot toward and away from each other.

5. The device of claim 1, wherein the foot retainer is positioned for measuring one or more of: a side bending movement, an horizontal bending movement, a vertical bending movement of the ankle joint.

6. The device of claim 1, wherein the inclined upper surface of the foot rest allows the foot at a horizontal angle for measurement of side-to-side or a vertical range of motion comprising dorsiflexion and plantar flexion.

7. The device of claim 1, wherein the inclined upper surface is inclined at approximately 40° with respect to the base.

8. The device of claim 1, wherein the one or more sensors are pressure sensors.

9. The device of claim 1, wherein the first and the second outputs are received by an acquisition data system.

10. The device of claim 1, further comprising an inclinometer configured to perform measurements of range of motion.

11. A device for measuring range of motion of an ankle, the device comprising:
a foot rest comprising an inclined upper surface;
a base;
at least one sidewall;
a foot retainer configured to secure a foot of a subject to the inclined upper surface of the foot rest for releasably receiving the foot of the subject;
a mounting plate, wherein the mounting plate is slidably mounted to the at least one sidewall;
an inclinometer, wherein the inclinometer is secured to an elongated rod;
a retaining bar, wherein the retaining bar is secured to the elongated rod adjacent its upper end, wherein the retaining bar is configured for positioning adjacent a calf muscle of the patient when the patient's foot is received in the foot retainer;
at least one strap disposed on the surface of the foot rest;
a first set of one or more sensors disposed on the surface of the foot rest;
a second set of one or more sensors disposed transversally and inferiorly along an internal surface of the at least one strap;
a first output component of the first set of the one or more sensors;
a second output of the second set of the one or more sensors;
an elongated rod having opposed upper end and lower end, wherein the lower end is rotatably mounted to the mounting plate;
a first set of one or more articulating joints configured to connect the inclined upper surface to the rear surface of the device;
wherein the first set of one or more articulating joint allows the inclined upper and the rear surface to pivot toward and away from each other.
a second set of one or more articulating joints configured to connect the rear surface of the device to the base;
wherein the second set of one or more articulating joint allows the rear surface and the base to pivot toward and away from each other;
an elongated rod having opposed upper end and lower end, wherein the lower end is rotatably mounted to a mounting plate; and

12. The device of claim 12, further comprising:
a measuring member, wherein the elongated rod and the measuring member form a goniometer.

13. A system for measuring range of motion of an ankle, the system comprising:
an acquisition data system;
a foot rest comprising an inclined upper surface;
a base;
at least one sidewall,
a foot retainer configured to secure a foot of a subject to the inclined upper surface of the foot rest for releasably receiving the foot of the subject;
a mounting plate, wherein the mounting plate is slidably mounted to the at least one sidewall;
an inclinometer, wherein the inclinometer is secured to an elongated rod;
a retaining bar, wherein the retaining bar is secured to the elongated rod adjacent its upper end, wherein the retaining bar is configured for positioning adjacent a calf muscle of the patient when the patient's foot is received in the foot retainer;
at least one strap disposed on the surface of the foot rest;
a first set of one or more sensors disposed on the surface of the foot rest;
a second set of one or more sensors disposed transversally and inferiorly along an internal surface of the at least one strap;
a first output component of the first set of the one or more sensors;
a second output of the second set of the one or more sensors; and
an elongated rod having opposed upper end and lower end, wherein the lower end is rotatably mounted to the mounting plate.

14. A method for measuring range of motion of an ankle, the method comprising:
placing a foot of a subject on a surface of a foot rest, wherein the foot rest comprises a base, at least one sidewall, and an inclined upper surface, and wherein a foot retainer is secured to the inclined upper surface of the foot rest for releasably receiving the foot of the subject;
measuring a side or horizontal bending movement of the ankle joint;
measuring a vertical bending movement of the ankle joint; and
obtaining information representative of one or more data sets from an acquisition data system during one or more physical actions performed by the subject.
